(19) United States
(12) Patent Application Publication
(10) Pub. No.: US 2025/0068668 A1
(43) Pub. Date: Feb. 27, 2025

(54) CLINICAL WORKFLOW EFFICIENCY USING LARGE LANGUAGE MODELS

(71) Applicant: Siemens Healthineers AG, Forchhein (DE)

(72) Inventors: Sasa Grbic, Plainsboro, NJ (US); Eli Gibson, Plainsboro, NJ (US); Oladimeji Farri, Upper Saddle River, NJ (US); Bogdan Georgescu, Princeton, NJ (US); Gianluca Paladini, Skillman, NJ (US); Puneet Sharma, Princeton Junction, NJ (US); Daphne Yu, Yardley, PA (US); Dorin Comaniciu, Princeton, NJ (US)

(21) Appl. No.: 18/453,697

(22) Filed: Aug. 22, 2023

(51) Int. Cl. G06F 16/34; G06F 40/40; G16H 10/60; G16H 30/20

(52) U.S. Cl. CPC G06F 16/345; G06F 40/40; G16H 10/60; G16H 30/20

(57) ABSTRACT

Systems and methods for generating a response summarizing patient data are provided. One or more prompts, comprising 1) patient data retrieved from one or more patient databases and 2) instructions, are received. A response summarizing the patient data is generated based on the instruction using a large language model. The response is output.

### TECHNICAL FIELD

The present invention relates generally to large language models, and in particular to improving clinical workflow efficiency using large language models.

### BACKGROUND

Healthcare providers typically store vast amounts of patient data in various patient databases. Such patient data is typically stored as unstructured data using different nomenclature. In the current clinical workflow, a clinician must manually search and retrieve all relevant patient information across the various patient databases, which is a time-consuming and inefficient task. Conventional approaches have been proposed to connect data retrieved from various patient databases for display in a patient dashboard. However, such conventional approaches are not scalable, as the semantic mapping across the various patient databases is often different for each clinical site, region, and country.

### BRIEF SUMMARY OF THE INVENTION

In accordance with one or more embodiments, systems and methods for generating a response summarizing patient data are provided. One or more prompts, comprising 1) patient data retrieved from one or more patient databases and 2) instructions, are received. A response summarizing the patient data is generated based on the instructions using a large language model. The response is output.

In one embodiment, the following steps are iteratively repeating for one or more iterations. One or more additional prompts comprising additional instructions are received. An additional response is generated based on the additional instructions. The additional response is output.

In one embodiment, the patient data comprises measurements and information automatically extracted from medical images using an artificial intelligence based system.

In one embodiment, the patient data comprises tabulated measurements. In one embodiment, the large language model is fine-tuned by: converting training tabulated data to token sequences; combining the token sequences with corresponding ground truth summaries; and fine-tuning the large language model based on the combined token sequences and corresponding ground truth summaries.

In one embodiment, the patient data is retrieved from a plurality of patient databases. The patient data may comprise unstructured data using different nomenclature. The one or more patient databases may comprise at least one of EHR (electronic health record), EMR (electronic medical record), PHR (personal health record), HIS (health information system), RIS (radiology information system), PACS (picture archiving and communication system), and LIMS (laboratory information management system).

In one embodiment, the large language model is constrained to a specific medical domain.

These and other advantages of the invention will be apparent to those of ordinary skill in the art by reference to the following detailed description and the accompanying drawings.

### BRIEF DESCRIPTION OF THE DRAWINGS

FIG. 1 shows a method for summarizing patient data retrieved from one or more patient databases, in accordance with one or more embodiments;

FIG. 2 shows a workflow for summarizing patient data retrieved from one or more patient databases, in accordance with one or more embodiments;

FIG. 3 shows a workflow for automatically generating a text-based response summarizing clinical measurements extracted from a medical image, in accordance with one or more embodiments;

FIG. 4 shows a workflow for automatically generating a text-based response summarizing tabulated patient data and medical images, in accordance with one or more embodiments;

FIG. 5 shows a workflow for automatically summarizing prior patient information, in accordance with one or more embodiments;

FIG. 6 shows a chat interface, in accordance with one or more embodiments;

FIG. 7 shows an exemplary artificial neural network that may be used to implement one or more embodiments;

FIG. 8 shows a convolutional neural network that may be used to implement one or more embodiments; and

FIG. 9 shows a high-level block diagram of a computer that may be used to implement one or more embodiments.

### DETAILED DESCRIPTION

The present invention generally relates to methods and systems for improving clinical workflow efficiency using large language models. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Patient data is typically stored across various patient databases as unstructured data using different nomenclature. Embodiments described herein provide for the summarization of such patient data using an LLM (large language model). The LLM provides for the interpretation and synthesis of summaries of such patient data, while also enabling conversational interaction with a clinician. Advantageously, embodiments described herein provide for improved operational efficiency and reduced cognitive load of clinicians.

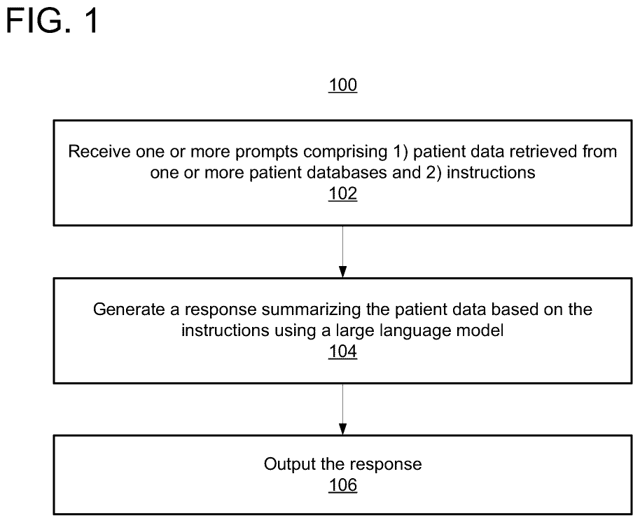

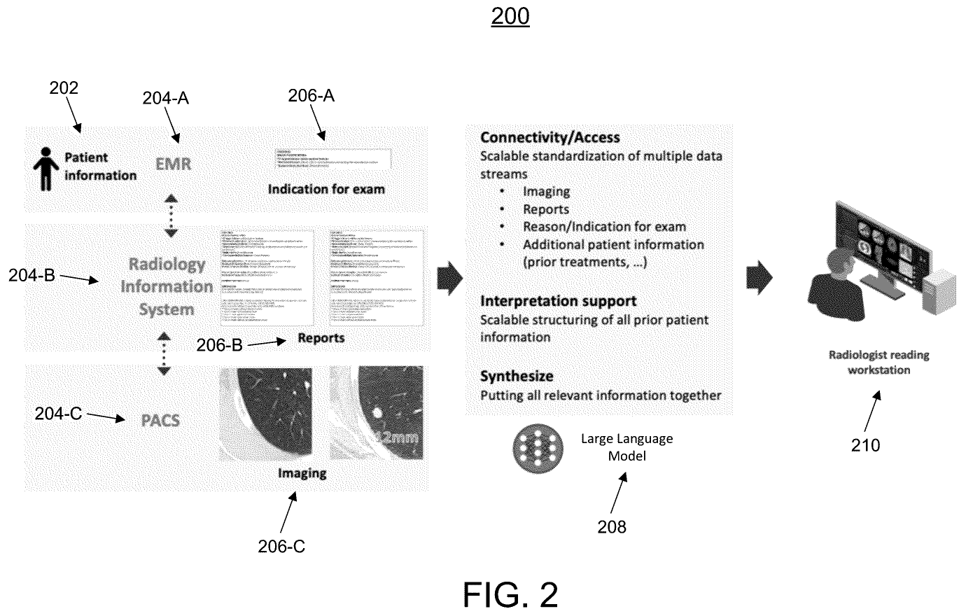

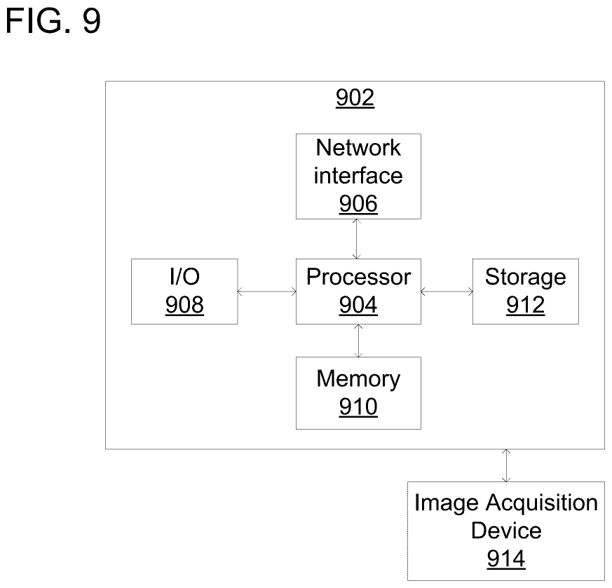

FIG. 1 shows a method 100 for summarizing patient data retrieved from one or more patient databases, in accordance with one or more embodiments. The steps of method 100 may be performed by one or more suitable computing devices, such as, e.g., computer 902 of FIG. 9. FIG. 2 shows a workflow 200 for summarizing patient data retrieved from one or more patient databases, in accordance with one or more embodiments. FIG. 1 and FIG. 2 will be described together.

At step 102 of FIG. 1, one or more prompts comprising 1) patient data retrieved from one or more patient databases and 2) instructions are received. A prompt refers to the input text to an LLM for generate a response. A prompt is typically provided by a user to enable the user to interact with the LLM. The one or more prompts may be received from a computing device (e.g., computer 902 of FIG. 9) with which a user is interacting.

The one or more prompts comprises patient data retrieved from one or more patient databases. The patient databases may include, for example, an EHR (electronic health record), EMR (electronic medical record), PHR (personal health record), HIS (health information system), RIS (radiology information system), PACS (picture archiving and communication system), LIMS (laboratory information management system), or any other database or system suitable for storing patient data.

In one embodiment, the patient data comprises text-based data of one or more patients, such as, e.g., medical records, laboratory reports, radiology reports, indications for imaging/examination, demographic information, administrative data, etc. stored across the one or more patient databases. In another embodiment, the patient data comprises medical images of one or more patients stored across the one or more patient databases. In this embodiment, the medical images may be automatically retrieved from the one or more patient databases but may additionally or alternatively be received as user input via the one or more prompts. The patient data may include any other suitable data of a patient. The patient data may be represented in the form of unstructured free text, tables, or any other suitable format using different nomenclature, for example, where the patient data is retrieved from a plurality of different patient databases. In one example, as shown in workflow 200 of FIG. 2, the patient data may be patient information 202 comprising indication for exam 206-A, reports 206-B, and imaging 206-C respectively retrieved from EMR 204-A, radiology information system 204-B, and PACS 204-C.

In one embodiment, the patient data comprises measurements and other information extracted from medical images. The extracted measurements and information may be manually extracted (e.g., by a radiologist or other clinician) and/or automatically from the medical images using AI (artificial intelligence) based systems performing one or more medical imaging analysis tasks (e.g., detection, classification, segmentation, etc.). The extracted measurements and information may be represented in one or more tables or in any other suitable format.

The one or more prompts also comprise an instruction to perform a medical task on the patient data. An instruction refers to guidelines or directions provided to guide the behavior and output of the LLM. An instruction may include commands, questions, constraints, requirements, or any other guideline or direction guiding the behavior and output of the LLM. The one or more prompts may include any other information for performing the medical task, such as, e.g., contextual data.

At step 104 of FIG. 1, in response to receiving the one or more prompts, a response summarizing the patient data is generated based on the instructions using a large language model. In one embodiment, the response comprises a text-based response (e.g., represented as sentences, phrases, or any other suitable format). However, the response may comprise a non-text-based response represented in any other suitable format (e.g., using an auxiliary machine learning based network). In one example, as shown in workflow 200 of FIG. 2, the large language model is large language model 208, which generates a response summarizing patient information 202. Lage language model 208 provides connectivity/access, interpretation support, and synthesis of summaries of patient information 202.

The LLM receives as input the one or more prompts and generates as output the response. The LLM may be any suitable pre-trained deep learning based LLM. For example, the LLM may be based on the transformer architecture, which uses a self-attention mechanism to capture long-range dependencies in text. One example of a transformer-based architecture is GPT (generative pre-training transformer), which has a multilayer transformer decoder architecture that may be pretrained to optimize the next token prediction task and then fine-tuned with labelled data for various downstream tasks. GPT-based LLMs may be trained using reinforcement learning with human feedback for performing various natural language processing tasks. Other exemplary transformer-based architectures include BLOOM (BigScience Large Open-science Open-access Multilingual Language Model) and BERT (Bidirectional Encoder Representations from Transfers).

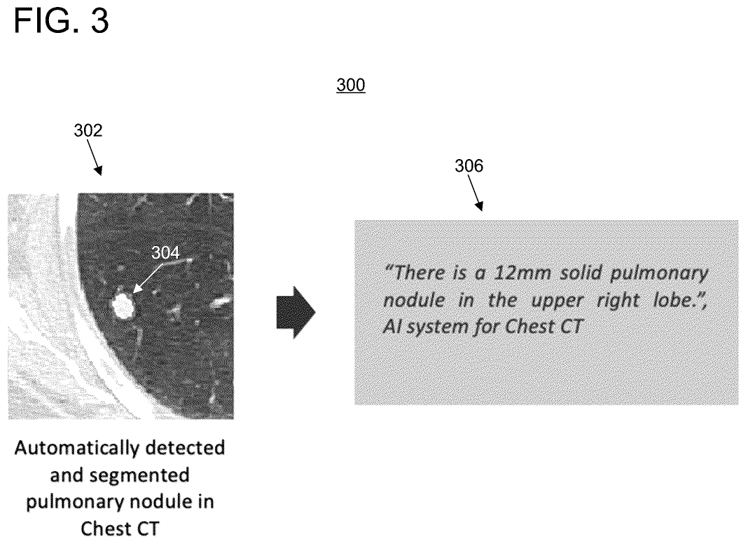

In one embodiment, the LLM is constrained to a specific medical domain. For example, the LLM may be constrained for the radiology use case for summarizing radiology-related patient data. To constrain the LLM to the radiology use case, the LLM may be updated (e.g., trained, retrained, or fine-tuned) using, e.g., clinical data and data extracted from medical images using AI-based systems. Such extracted data may include, e.g., clinical measurements (e.g., diameters, volumes, distances, etc.), anatomical locations, detections, etc. The constrained LLM may automatically generate a response as output based on such clinical measurements. FIG. 3 shows a workflow 300 for automatically generating a text-based response summarizing clinical measurements extracted from a medical image, in accordance with one or more embodiments. In workflow 300, one or more AI-based systems automatically detect and segment pulmonary nodule 304 is chest CT (computed tomography) image 302. An LLM, constrained for the radiology use case, receives as input clinical measurements and other findings of the AI-based systems (as patient data) and generates as output a text-based response 306 summarizing the clinical measurements and other findings as follows: “There is a 12 mm (millimeter) solid pulmonary nodule in the upper right lobe.”

Referring back to step 104 of FIG. 1, in one embodiment, the LLM may leverage tabulated data output from AI-based systems. In one embodiment, the LLM may be constrained by supervised fine-tuning to convert training tabulated data to text files or token sequences, which are combined with corresponding ground truth user-generated summaries to form input-output pairs. For a sequence of token on measurements x¹, . . . , x^{m} during fine-tuning, the hidden state from the decoder block hₗ^{m} is passed to a softmax layer with θ as the learned parameters to predict summary tokens y as follows:

$$P ( y \vert {x}^{1} , … , {x}^{m} ) = softmax ( {h}_{l}^{m} θ ) \qquad Equation ( l )$$

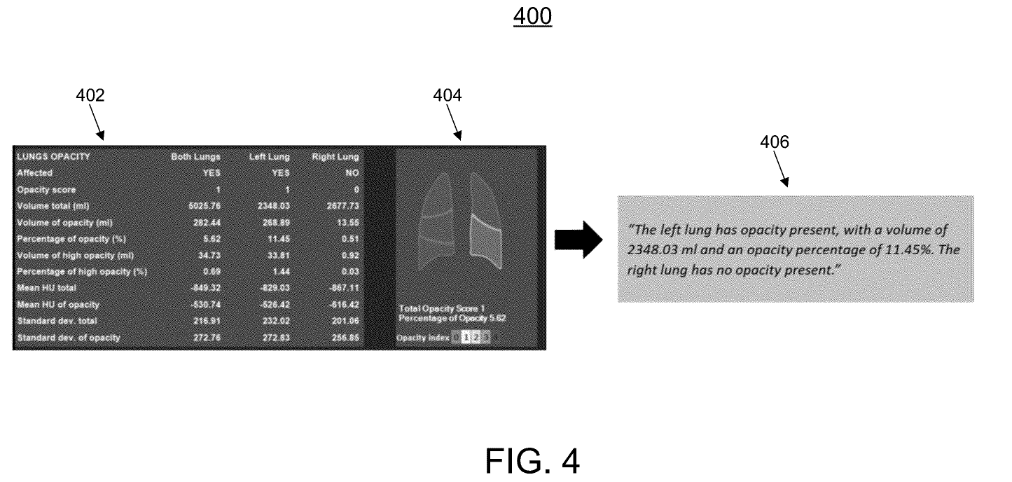

FIG. 4 shows a workflow 400 for automatically generating a text-based response summarizing tabulated patient data and medical images, in accordance with one or more embodiments. Workflow 400 comprises tabulated patient data 402 and medical image 404. Tabulated patient data 402 comprises clinical measurements generated by an AI-based system. Tabulated results 402 are transformed into a flat sequence of entries separate by a delimiter. Medical image 404 comprises an opacity index or score for each portion of the lungs and a percentage of opacity. Medical image 404 is encoded to embeddings through an image-specific embedding function (e.g., another machine learning based network). The embeddings are aligned with the text embedding function used by the LLM. The LLM, constrained for the radiology use case, receives as input the sequence of entries transformed from tabulated patient data 402 and the embeddings of medical image 404 (as patient data) and generates as output a text-based response 406 summarizing the tabulated patient data 402 and medical image 404 as follows: “the left lung has opacity present, with a volume of 2348.03 ml (milliliters) and an opacity percentage of 11.45%. The right lung has no opacity present.”

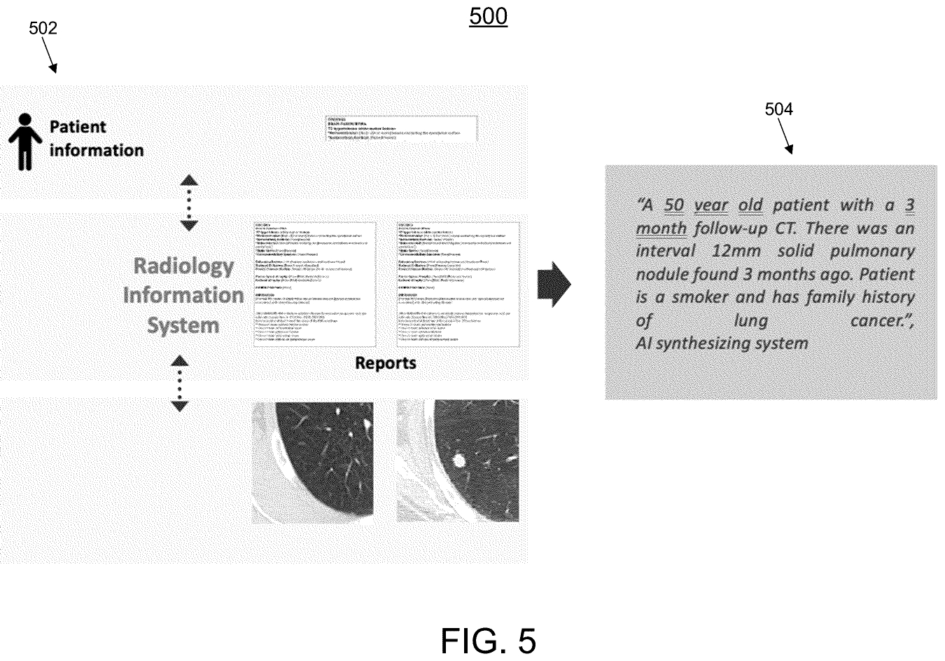

Referring back to step 104 of FIG. 1, in one embodiment, the LLM may be leveraged to summarize all prior patient information in a concise manner, e.g., before the radiologist reads the case. FIG. 5 shows a workflow 500 for automatically summarizing prior patient information, in accordance with one or more embodiments. In workflow 500, patient information 502 to be summarized, including extracted clinical measurements, reports, and medical images, is stored in a radiology information system. An LLM, constrained for the radiology use case, receives as input patient information 502 and generates as output a text-based response 504 as follows: “A 50 year old patient with a 3 month follow-up CT. There was an interval 12 mm (millimeter) solid pulmonary nodule found 3 months ago. Patient is a smoker and has family history of lung cancer.” The medical images are encoded to embeddings through an image-specific embedding function (e.g., another machine learning based network).

Referring back to FIG. 1, at step 106, the response is output. For example, the response can be output by displaying the response on a display device of a computer system (e.g., computer 902 of FIG. 9), storing the response on a memory or storage of a computer system, or by transmitting the response to a remote computer system. In one example, as shown in workflow 200 of FIG. 2, the response output by LLM 208 is displayed to a radiologist (or any other user) on radiologist reading workstation 210.

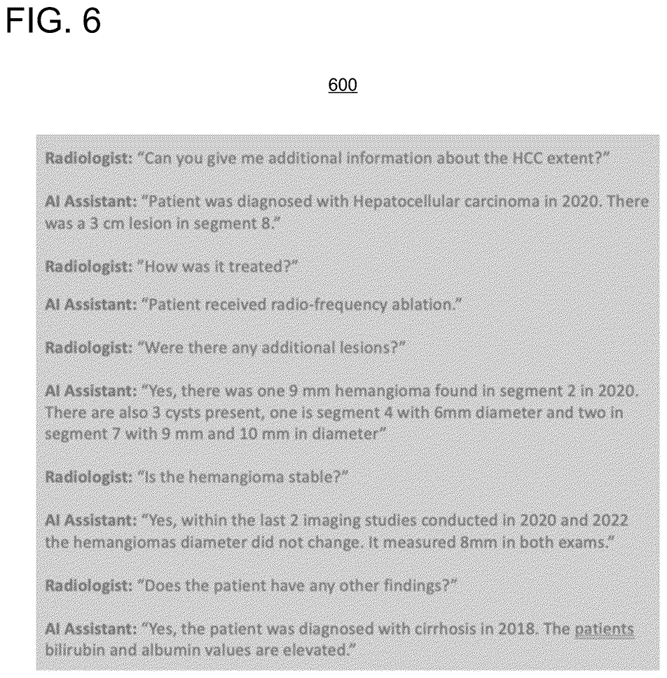

In one embodiment, the user can ask additional questions to the LLM during case interpretation through, for example, a text-based or voice-based chat interface. For example, in response to the output response, one or more additional prompts comprising additional instructions may be received from the user and an additional response may be generated based on the additional instructions using the large language model and output (e.g., displayed) to the user. This may be repeated for one or more iterations to provide for a chat interface with the LLM. This allows for the efficient presentation of information that is salient based on the user's understanding of the clinical scenario. FIG. 6 shows a chat interface 600, in accordance with one or more embodiments. In chat interface 600, a radiologist inputs prompts comprising instructions (e.g., questions) to an LLM-based AI assistant and the AI assistant provides responses to the instructions.

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems utilizing trained machine learning based models, as well as with respect to methods and systems for training machine learning based models. Features, advantages or alternative embodiments herein can be assigned to the other claimed objects and vice versa. In other words, claims for methods and systems for training a machine learning based model can be improved with features described or claimed in context of the methods and systems for utilizing a trained machine learning based model, and vice versa.

In particular, the trained machine learning based models applied in embodiments described herein can be adapted by the methods and systems for training the machine learning based models. Furthermore, the input data of the trained machine learning based model can comprise advantageous features and embodiments of the training input data, and vice versa. Furthermore, the output data of the trained machine learning based model can comprise advantageous features and embodiments of the output training data, and vice versa.

In general, a trained machine learning based model mimics cognitive functions that humans associate with other human minds. In particular, by training based on training data, the trained machine learning based model is able to adapt to new circumstances and to detect and extrapolate patterns.

In general, parameters of a machine learning based model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based model can be adapted iteratively by several steps of training.

In particular, a trained machine learning based model can comprise a neural network, a support vector machine, a decision tree, and/or a Bayesian network, and/or the trained machine learning based model can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

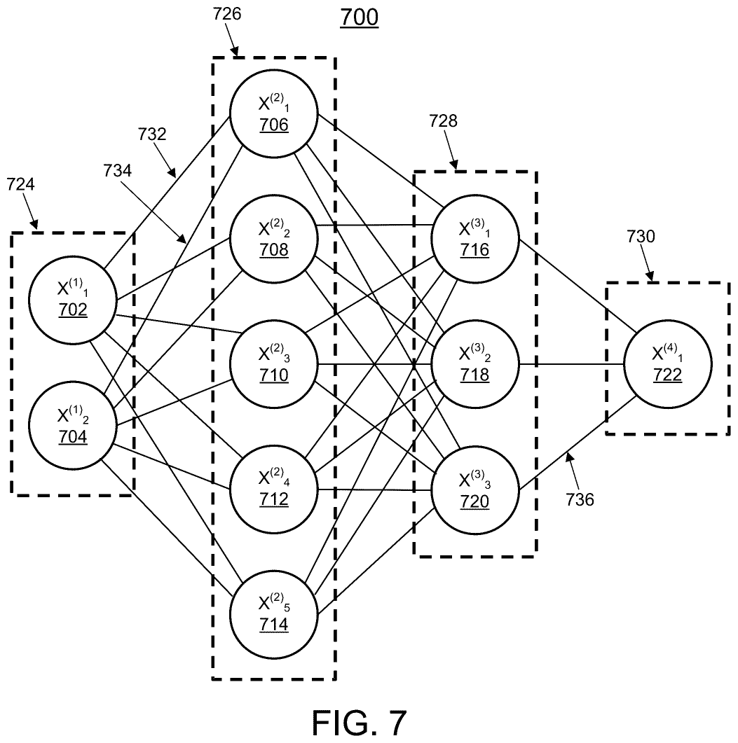

FIG. 7 shows an embodiment of an artificial neural network 700, in accordance with one or more embodiments. Alternative terms for “artificial neural network” are “neural network”, “artificial neural net” or “neural net”. Machine learning networks described herein, such as, e.g., the large language model utilized at step 104 of FIG. 1 and the large language model 208 of FIG. 2, may be implemented using artificial neural network 700.

The artificial neural network 700 comprises nodes 702-722 and edges 732, 734, . . . , 736, wherein each edge 732, 734, . . . , 736 is a directed connection from a first node 702-722 to a second node 702-722. In general, the first node 702-722 and the second node 702-722 are different nodes 702-722, it is also possible that the first node 702-722 and the second node 702-722 are identical. For example, in FIG. 7, the edge 732 is a directed connection from the node 702 to the node 706, and the edge 734 is a directed connection from the node 704 to the node 706. An edge 732, 734, . . . , 736 from a first node 702-722 to a second node 702-722 is also denoted as “ingoing edge” for the second node 702-722 and as “outgoing edge” for the first node 702-722.

In this embodiment, the nodes 702-722 of the artificial neural network 700 can be arranged in layers 724-730, wherein the layers can comprise an intrinsic order introduced by the edges 732, 734, . . . , 736 between the nodes 702-722. In particular, edges 732, 734, . . . , 736 can exist only between neighboring layers of nodes. In the embodiment shown in FIG. 7, there is an input layer 724 comprising only nodes 702 and 704 without an incoming edge, an output layer 730 comprising only node 722 without outgoing edges, and hidden layers 726, 728 in-between the input layer 724 and the output layer 730. In general, the number of hidden layers 726, 728 can be chosen arbitrarily. The number of nodes 702 and 704 within the input layer 724 usually relates to the number of input values of the neural network 700, and the number of nodes 722 within the output layer 730 usually relates to the number of output values of the neural network 700.

In particular, a (real) number can be assigned as a value to every node 702-722 of the neural network 700. Here, x⁽ⁿ⁾ᵢ denotes the value of the i-th node 702-722 of the n-th layer 724-730. The values of the nodes 702-722 of the input layer 724 are equivalent to the input values of the neural network 700, the value of the node 722 of the output layer 730 is equivalent to the output value of the neural network 700. Furthermore, each edge 732, 734, . . . , 736 can comprise a weight being a real number, in particular, the weight is a real number within the interval [−1, 1] or within the interval [0, 1]. Here, w^{(m,n)}_{i,j} denotes the weight of the edge between the i-th node 702-722 of the m-th layer 724-730 and the j-th node 702-722 of the n-th layer 724-730. Furthermore, the abbreviation w⁽ⁿ⁾_{i,j} is defined for the weight w^{(n,n+1)}_{i,j}.

In particular, to calculate the output values of the neural network 700, the input values are propagated through the neural network. In particular, the values of the nodes 702-722 of the (n+1)-th layer 724-730 can be calculated based on the values of the nodes 702-722 of the n-th layer 724-730 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 724 are given by the input of the neural network 700, wherein values of the first hidden layer 726 can be calculated based on the values of the input layer 724 of the neural network, wherein values of the second hidden layer 728 can be calculated based in the values of the first hidden layer 726, etc.

In order to set the values w^{(m,n)}_{i,j} for the edges, the neural network 700 has to be trained using training data. In particular, training data comprises training input data and training output data (denoted as ti). For a training step, the neural network 700 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 700 (backpropagation algorithm). In particular, the weights are changed according to

$${w}_{i , j}^{′ ( n )} = {w}_{i , j}^{( n )} - y \cdot {δ}_{j}^{( n )} \cdot {x}_{i}^{( n )}$$

wherein γ is a learning rate, and the numbers δ⁽ⁿ⁾ⱼ can be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 730, wherein f′ is the first derivative of the activation function, and y⁽ⁿ⁺¹⁾ⱼ is the comparison training value for the j-th node of the output layer 730.

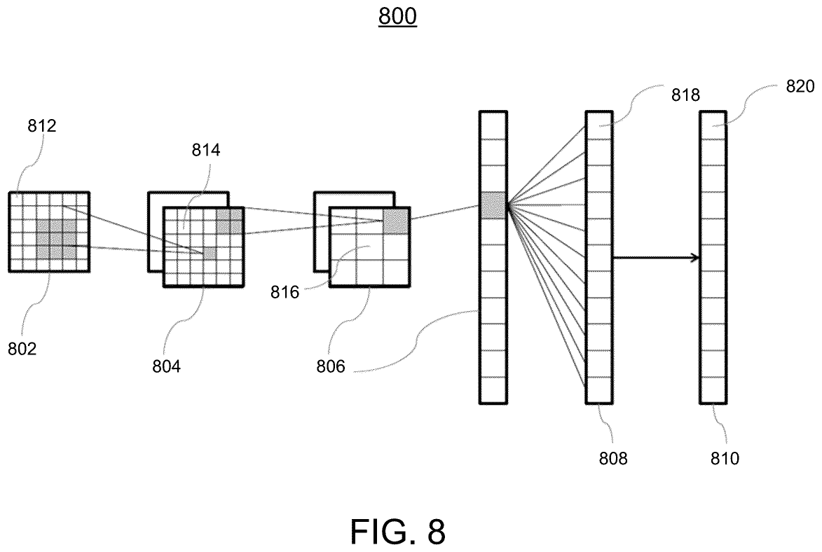

FIG. 8 shows a convolutional neural network 800, in accordance with one or more embodiments. Machine learning networks described herein, such as, e.g., the large language model utilized at step 104 of FIG. 1 and the large language model 208 of FIG. 2, may be implemented using convolutional neural network 800.

In the embodiment shown in FIG. 8, the convolutional neural network comprises 800 an input layer 802, a convolutional layer 804, a pooling layer 806, a fully connected layer 808, and an output layer 810. Alternatively, the convolutional neural network 800 can comprise several convolutional layers 804, several pooling layers 806, and several fully connected layers 808, as well as other types of layers. The order of the layers can be chosen arbitrarily, usually fully connected layers 808 are used as the last layers before the output layer 810.

In particular, within a convolutional neural network 800, the nodes 812-820 of one layer 802-810 can be considered to be arranged as a d-dimensional matrix or as a d-dimensional image. In particular, in the two-dimensional case the value of the node 812-820 indexed with i and j in the n-th layer 802-810 can be denoted as x⁽ⁿ⁾_{[i,j]}. However, the arrangement of the nodes 812-820 of one layer 802-810 does not have an effect on the calculations executed within the convolutional neural network 800 as such, since these are given solely by the structure and the weights of the edges.

In particular, a convolutional layer 804 is characterized by the structure and the weights of the incoming edges forming a convolution operation based on a certain number of kernels. In particular, the structure and the weights of the incoming edges are chosen such that the values x⁽ⁿ⁾ₖ of the nodes 814 of the convolutional layer 804 are calculated as a convolution x⁽ⁿ⁾ₖ=Kₖ*x⁽ⁿ⁻¹⁾ based on the values x⁽ⁿ⁻¹⁾ of the nodes 812 of the preceding layer 802, where the convolution * is defined in the two-dimensional case as

$${x}_{k}^{( n )} [ i , j ] = ( {K}_{k} * {x}^{( n - 1 )} ) [ i , j ] = {\sum }_{{i}^{1}} {\sum }_{{j}^{1}} {K}_{k} [ {i}^{1} , {j}^{1} ] \cdot {x}^{( n - 1 )} [ i - {i}^{1} , j - {j}^{1} ] .$$

Here the k-th kernel Kₖ is a d-dimensional matrix (in this embodiment a two-dimensional matrix), which is usually small compared to the number of nodes 812-818 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 812-820 in the respective layer 802-810. In particular, for a convolutional layer 804, the number of nodes 814 in the convolutional layer is equivalent to the number of nodes 812 in the preceding layer 802 multiplied with the number of kernels.

If the nodes 812 of the preceding layer 802 are arranged as a d-dimensional matrix, using a plurality of kernels can be interpreted as adding a further dimension (denoted as “depth” dimension), so that the nodes 814 of the convolutional layer 804 are arranged as a (d+1)-dimensional matrix. If the nodes 812 of the preceding layer 802 are already arranged as a (d+1)-dimensional matrix comprising a depth dimension, using a plurality of kernels can be interpreted as expanding along the depth dimension, so that the nodes 814 of the convolutional layer 804 are arranged also as a (d+1)-dimensional matrix, wherein the size of the (d+1)-dimensional matrix with respect to the depth dimension is by a factor of the number of kernels larger than in the preceding layer 802.

The advantage of using convolutional layers 804 is that spatially local correlation of the input data can exploited by enforcing a local connectivity pattern between nodes of adjacent layers, in particular by each node being connected to only a small region of the nodes of the preceding layer.

In embodiment shown in FIG. 8, the input layer 802 comprises 36 nodes 812, arranged as a two-dimensional 6×6 matrix. The convolutional layer 804 comprises 72 nodes 814, arranged as two two-dimensional 6×6 matrices, each of the two matrices being the result of a convolution of the values of the input layer with a kernel. Equivalently, the nodes 814 of the convolutional layer 804 can be interpreted as arranges as a three-dimensional 6×6×2 matrix, wherein the last dimension is the depth dimension.

A pooling layer 806 can be characterized by the structure and the weights of the incoming edges and the activation function of its nodes 816 forming a pooling operation based on a non-linear pooling function f. For example, in the two dimensional case the values x⁽ⁿ⁾ of the nodes 816 of the pooling layer 806 can be calculated based on the values x⁽ⁿ⁻¹⁾ of the nodes 814 of the preceding layer 804 as

$${x}^{( n )} [ i , j ] = f ( {x}^{( n - 1 )} [ {id}_{1} , j {d}_{2} ] , … , {x}^{( n - 1 )} [ {id}_{1} + {d}_{1} - 1 , j {d}_{2} + {d}_{2} - 1 ] )$$

In other words, by using a pooling layer 806, the number of nodes 814, 816 can be reduced, by replacing a number d1·d2 of neighboring nodes 814 in the preceding layer 804 with a single node 816 being calculated as a function of the values of said number of neighboring nodes in the pooling layer. In particular, the pooling function f can be the max-function, the average or the L2-Norm. In particular, for a pooling layer 806 the weights of the incoming edges are fixed and are not modified by training.

The advantage of using a pooling layer 806 is that the number of nodes 814, 816 and the number of parameters is reduced. This leads to the amount of computation in the network being reduced and to a control of overfitting.

In the embodiment shown in FIG. 8, the pooling layer 806 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 808 can be characterized by the fact that a majority, in particular, all edges between nodes 816 of the previous layer 806 and the nodes 818 of the fully-connected layer 808 are present, and wherein the weight of each of the edges can be adjusted individually.

In this embodiment, the nodes 816 of the preceding layer 806 of the fully-connected layer 808 are displayed both as two-dimensional matrices, and additionally as non-related nodes (indicated as a line of nodes, wherein the number of nodes was reduced for a better presentability). In this embodiment, the number of nodes 818 in the fully connected layer 808 is equal to the number of nodes 816 in the preceding layer 806. Alternatively, the number of nodes 816, 818 can differ.

Furthermore, in this embodiment, the values of the nodes 820 of the output layer 810 are determined by applying the Softmax function onto the values of the nodes 818 of the preceding layer 808. By applying the Softmax function, the sum the values of all nodes 820 of the output layer 810 is 1, and all values of all nodes 820 of the output layer are real numbers between 0 and 1.

A convolutional neural network 800 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 800 can be trained based on the backpropagation algorithm. For preventing overfitting, methods of regularization can be used, e.g. dropout of nodes 812-820, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 902 that may be used to implement systems, apparatus, and methods described herein is depicted in FIG. 9. Computer 902 includes a processor 904 operatively coupled to a data storage device 912 and a memory 910. Processor 904 controls the overall operation of computer 902 by executing computer program instructions that define such operations. The computer program instructions may be stored in data storage device 912, or other computer readable medium, and loaded into memory 910 when execution of the computer program instructions is desired. Thus, the method and workflow steps or functions of FIG. 1 or 2 can be defined by the computer program instructions stored in memory 910 and/or data storage device 912 and controlled by processor 904 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 904 executes the method and workflow steps or functions of FIG. 1 or 2. Computer 902 may also include one or more network interfaces 906 for communicating with other devices via a network. Computer 902 may also include one or more input/output devices 908 that enable user interaction with computer 902 (e.g., display, keyboard, mouse, speakers, buttons, etc.).

Processor 904 may include both general and special purpose microprocessors, and may be the sole processor or one of multiple processors of computer 902. Processor 904 may include one or more central processing units (CPUs), for example. Processor 904, data storage device 912, and/or memory 910 may include, be supplemented by, or incorporated in, one or more application-specific integrated circuits (ASICs) and/or one or more field programmable gate arrays (FPGAs).

Data storage device 912 and memory 910 each include a tangible non-transitory computer readable storage medium. Data storage device 912, and memory 910, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 908 may include peripherals, such as a printer, scanner, display screen, etc. For example, input/output devices 908 may include a display device such as a cathode ray tube (CRT) or liquid crystal display (LCD) monitor for displaying information to the user, a keyboard, and a pointing device such as a mouse or a trackball by which the user can provide input to computer 902.

An image acquisition device 914 can be connected to the computer 902 to input image data (e.g., medical images) to the computer 902. It is possible to implement the image acquisition device 914 and the computer 902 as one device. It is also possible that the image acquisition device 914 and the computer 902 communicate wirelessly through a network. In a possible embodiment, the computer 902 can be located remotely with respect to the image acquisition device 914.

Any or all of the systems and apparatus discussed herein may be implemented using one or more computers such as computer 902.

One skilled in the art will recognize that an implementation of an actual computer or computer system may have other structures and may contain other components as well, and that FIG. 9 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
receiving one or more prompts comprising 1) patient data retrieved from one or more patient databases and 2) instructions;
generating a response summarizing the patient data based on the instructions using a large language model; and
outputting the response.

2. The computer-implemented method of claim 1, further comprising:
iteratively repeating, for one or more iterations, the following steps:
receiving one or more additional prompts comprising additional instructions;
generating an additional response based on the additional instructions; and
outputting the additional response.

3. The computer-implemented method of claim 1, wherein the patient data comprises measurements and information automatically extracted from medical images using an artificial intelligence based system.

4. The computer-implemented method of claim 1, wherein the patient data comprises tabulated measurements.

5. The computer-implemented method of claim 4, wherein the large language model is fine-tuned by:
converting training tabulated data to token sequences;
combining the token sequences with corresponding ground truth summaries; and
fine-tuning the large language model based on the combined token sequences and corresponding ground truth summaries.

6. The computer-implemented method of claim 1, wherein the patient data is retrieved from a plurality of patient databases.

7. The computer-implemented method of claim 1, wherein the patient data comprises unstructured data using different nomenclature.

8. The computer-implemented method of claim 1, wherein the one or more patient databases comprise at least one of EHR (electronic health record), EMR (electronic medical record), PHR (personal health record), HIS (health information system), RIS (radiology information system), PACS (picture archiving and communication system), and LIMS (laboratory information management system).

9. The computer-implemented method of claim 1, wherein the large language model is constrained to a specific medical domain.

10. An apparatus comprising:
means for receiving one or more prompts comprising 1) patient data retrieved from one or more patient databases and 2) instructions;
means for generating a response summarizing the patient data based on the instructions using a large language model; and
means for outputting the response.

11. The apparatus of claim 10, further comprising:
means for iteratively repeating, for one or more iterations, the following steps:
means for receiving one or more additional prompts comprising additional instructions;
means for generating an additional response based on the additional instructions; and
means for outputting the additional response.

12. The apparatus of claim 10, wherein the patient data comprises measurements and information automatically extracted from medical images using an artificial intelligence based system.

13. The apparatus of claim 10, wherein the patient data comprises tabulated measurements.

14. The apparatus of claim 13, wherein the large language model is fine-tuned by:
means for converting training tabulated data to token sequences;
means for combining the token sequences with corresponding ground truth summaries; and
means for fine-tuning the large language model based on the combined token sequences and corresponding ground truth summaries.

15. A non-transitory computer readable medium storing computer program instructions, the computer program instructions when executed by a processor cause the processor to perform operations comprising:
receiving one or more prompts comprising 1) patient data retrieved from one or more patient databases and 2) instructions;
generating a response summarizing the patient data based on the instructions using a large language model; and
outputting the response.

16. The non-transitory computer readable medium of claim 15, further comprising:
iteratively repeating, for one or more iterations, the following operations:
receiving one or more additional prompts comprising additional instructions;
generating an additional response based on the additional instructions; and
outputting the additional response.

17. The non-transitory computer readable medium of claim 15, wherein the patient data is retrieved from a plurality of patient databases.

18. The non-transitory computer readable medium of claim 15, wherein the patient data comprises unstructured data using different nomenclature.

19. The non-transitory computer readable medium of claim 15, wherein the one or more patient databases comprise at least one of EHR (electronic health record), EMR (electronic medical record), PHR (personal health record), HIS (health information system), RIS (radiology information system), PACS (picture archiving and communication system), and LIMS (laboratory information management system).

20. The non-transitory computer readable medium of claim 15, wherein the large language model is constrained to a specific medical domain.
